(19) United States
(12) Patent Application Publication
(10) Pub. No.: US 2005/0203412 A1
(43) Pub. Date: Sep. 15, 2005

(54) Method of controlling ultrasonic probe and ultrasonic diagnostic apparatus

(75) Inventor: Shinichi Amemiya, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/075,096

(22) Filed: Mar. 8, 2005

(30) Foreign Application Priority Data
Mar. 12, 2004 (JP) 2004-069858

(51) Int. Cl. A61B 8/14

(52) U.S. Cl. 600/459

(57) ABSTRACT

A method and apparatus for forming a desired ultrasonic beam even when an acoustic line is directed obliquely with respect to an axis of symmetry of a transducer in-aperture array, when an acoustic line is directed obliquely to one side with respect to an axis of symmetry of a transducer in-aperture array, a weight assigned to the transducers is set to be asymmetric with respect to the axis of symmetry.

### BACKGROUND OF THE INVENTION

The present invention relates to a method of controlling an ultrasonic probe and an ultrasonic diagnostic apparatus, and more particularly to a method of controlling an ultrasonic probe and an ultrasonic diagnostic apparatus capable of forming a desired ultrasonic beam even when an acoustic line is directed obliquely with respect to an axis of symmetry of a transducer in-aperture array (i.e., an arrangement of transducers that actually work in transmission or reception of ultrasound. When the aperture is smaller than the length of a transducer array in an ultrasonic probe, the aperture corresponds to a portion of the transducer array in the ultrasonic probe).

Conventionally, there is known an ultrasonic diagnostic apparatus intended for magnification of a field of view by conducting a combination scan of linear and sector scan schemes or convex and sector scan schemes (for example, see Patent Document 1).

[Patent Document 1] Japanese Patent Application Laid Open No. 2000-300560.

In the conventional ultrasonic diagnostic apparatus, the transducers are assigned a sidelobe suppressing weight as exemplarily shown in FIG. 3 to suppress a sidelobe. The sidelobe suppressing weight is defined to be symmetric with respect to an axis of symmetry of a transducer in-aperture array, and this causes no concern in a linear or convex scan scheme.

However, when a sector scan scheme is combined for magnification of a field of view, since the acoustic line is obliquely directed to, for example, the left side with respect to the axis of symmetry of the transducer in-aperture array as shown in FIG. 2, a distance Dr from a transducer 10r, which lies on the right side of the axis of symmetry (at the position of a transducer 10c) of the transducer in-aperture array, to a focusfo is increased relative to a distance D1 from a transducer 10l lying on the left side to the focus fo. Thus, the ultrasound decay value of the transducers becomes asymmetric with respect to the axis of symmetry of the transducer array, as exemplarily shown in FIG. 4.

As a result, the weight assigned to the transducers becomes asymmetric as viewed from the focus fo, as exemplarily shown in FIG. 5, which leads to a problem that a desired ultrasonic beam cannot be formed.

### SUMMARY OF THE INVENTION

It is therefore an object of the present invention to provide a method of controlling an ultrasonic probe and an ultrasonic diagnostic apparatus capable of forming a desired ultrasonic beam even when an acoustic line is directed obliquely with respect to an axis of symmetry of a transducer in-aperture array.

In its first aspect, the present invention provides a method of controlling an ultrasonic probe characterized in comprising: setting a transmission delay time and a reception delay time to be asymmetric for transducers lying at positions symmetric with respect to an axis of symmetry of a transducer in-aperture array to direct an acoustic line obliquely with respect to said axis of symmetry; and setting at least one of a transmission power and a reception gain corresponding to said transducers to be asymmetric.

According to the method of controlling an ultrasonic probe of the first aspect, when the decay value is asymmetric with respect to the axis of symmetry of the transducer in-aperture array, the weight assigned to the transducers is set to be asymmetric; therefore, it is possible by such setting to make the weight for the transducers symmetric as viewed from the focus so as to mutually cancel asymmetry, thus forming a desired ultrasonic beam.

In its second aspect, the present invention provides the method of controlling an ultrasonic probe having the aforementioned configuration, characterized in that: at least one of a transmission power and a reception gain is decreased for a transducer having a longer transmission delay time and reception delay time.

According to the method of controlling an ultrasonic probe of the second aspect, when the decay value is asymmetric with respect to the axis of symmetry of the transducer in-aperture array, the weight assigned to the transducers is set to be asymmetric to cancel the asymmetry; therefore, it is possible to make the weight for the transducers symmetric as viewed from the focus, thus forming a desired ultrasonic beam.

In its third aspect, the present invention provides the method of controlling an ultrasonic probe having the aforementioned configuration, characterized in that: the degree of asymmetry is increased for a higher frequency of ultrasound.

When the decay value is asymmetric with respect to the axis of symmetry of the transducer in-aperture array, the asymmetry is larger for a higher frequency of ultrasound, as will be discussed later.

According to the method of controlling an ultrasonic probe of the third aspect, the degree of asymmetry of the weight assigned to the transducers is therefore increased for a higher frequency of ultrasound. Thus, asymmetry of the decay value can be canceled, and it is possible to make the weight for the transducers symmetric as viewed from the focus, thus forming a desired ultrasonic beam.

In its fourth aspect, the present invention provides the method of controlling an ultrasonic probe having the aforementioned configuration, characterized in that: the degree of asymmetry is increased for a larger angle of the oblique direction of an acoustic line with respect to said axis of symmetry.

When the decay value is asymmetric with respect to the axis of symmetry of the transducer in-aperture array, the asymmetry is larger for a larger angle of the oblique direction of an acoustic line, as will be discussed later.

According to the method of controlling an ultrasonic probe of the fourth aspect, the degree of asymmetry of the weight assigned to the transducers is therefore increased for a larger angle of the oblique direction of an acoustic line with respect to said axis of symmetry. The asymmetry of the decay value is thus canceled, and it is possible to make the weight for the transducers symmetric as viewed from the focus, thus forming a desired ultrasonic beam.

In its fifth aspect, the present invention provides the method of controlling an ultrasonic probe having the aforementioned configuration, characterized in that: said transmission power is controlled by controlling the amplitude of a transducer driving pulse.

According to the method of controlling an ultrasonic probe of the fifth aspect, the weight can be imparted depending upon the magnitude of the amplitude of a transducer driving pulse applied to the transducers.

In its sixth aspect, the present invention provides the method of controlling an ultrasonic probe having the aforementioned configuration, characterized in that: said transmission power is controlled by controlling the pulse width of the transducer driving pulse.

According to the method of controlling an ultrasonic probe of the sixth aspect, the weight can be imparted depending upon the length of the pulse width of the transducer driving pulse applied to the transducers.

In its seventh aspect, the present invention provides the method of controlling an ultrasonic probe having the aforementioned configuration, characterized in that: said ultrasonic probe is a linear ultrasonic probe having transducers arranged in a straight line, and said linear ultrasonic probe is used to conduct a virtual convex scan.

According to the method of controlling an ultrasonic probe of the seventh aspect, the present invention can be applied to a combination scan of linear and sector scan schemes.

In its eighth aspect, the present invention provides the method of controlling an ultrasonic probe having the aforementioned configuration, characterized in that: said ultrasonic probe is a convex ultrasonic probe having transducers arranged in a circular arc, and said convex ultrasonic probe is used to conduct an offset convex scan.

According to the method of controlling an ultrasonic probe of the eighth aspect, the present invention can be applied to a combination scan of convex and sector scan schemes.

In its ninth aspect, the present invention provides an ultrasonic diagnostic apparatus characterized in comprising: an ultrasonic probe having a plurality of transducers arranged therein; acoustic line direction controlling means for setting a transmission delay time and a reception delay time to be asymmetric for transducers lying at positions symmetric with respect to an axis of symmetry of a transducer in-aperture array to direct an acoustic line obliquely with respect to said axis of symmetry; and weight controlling means for setting at least one of a transmission power and a reception gain corresponding to said transducers to be asymmetric.

According to the ultrasonic diagnostic apparatus of the ninth aspect, the method of controlling an ultrasonic probe of the first aspect can be suitably implemented.

In its tenth aspect, the present invention provides the ultrasonic diagnostic apparatus having the aforementioned configuration, characterized in that: said weight controlling means decreases at least one of a transmission power and a reception gain for a transducer having a longer transmission delay time and reception delay time.

According to the ultrasonic diagnostic apparatus of the tenth aspect, the method of controlling an ultrasonic probe of the second aspect can be suitably implemented.

In its eleventh aspect, the present invention provides the ultrasonic diagnostic apparatus having the aforementioned configuration, characterized in that: said weight controlling means increases the degree of asymmetry for a higher frequency of ultrasound.

According to the ultrasonic diagnostic apparatus of the eleventh aspect, the method of controlling an ultrasonic probe of the third aspect can be suitably implemented.

In its twelfth aspect, the present invention provides the ultrasonic diagnostic apparatus having the aforementioned configuration, characterized in that: said weight controlling means increases the degree of asymmetry for a larger angle of the oblique direction of an acoustic line with respect to said axis of symmetry.

According to the ultrasonic diagnostic apparatus of the twelfth aspect, the method of controlling an ultrasonic probe of the fourth aspect can be suitably implemented.

In its thirteenth aspect, the present invention provides the ultrasonic diagnostic apparatus having the aforementioned configuration, characterized in that: said transmission power is controlled by controlling the amplitude of a transducer driving pulse.

According to the ultrasonic diagnostic apparatus of the thirteenth aspect, the method of controlling an ultrasonic probe of the fifth aspect can be suitably implemented.

In its fourteenth aspect, the present invention provides the ultrasonic diagnostic apparatus having the aforementioned configuration, characterized in that: said transmission power is controlled by controlling the pulse width of the transducer driving pulse.

According to the ultrasonic diagnostic apparatus of the fourteenth aspect, the method of controlling an ultrasonic probe of the sixth aspect can be suitably implemented.

In its fifteenth aspect, the present invention provides the ultrasonic diagnostic apparatus having the aforementioned configuration, characterized in that: said ultrasonic probe is a linear ultrasonic probe having transducers arranged in a straight line.

According to the ultrasonic diagnostic apparatus of the fifteenth aspect, the method of controlling an ultrasonic probe of the seventh aspect can be suitably implemented.

In its sixteenth aspect, the present invention provides the ultrasonic diagnostic apparatus having the aforementioned configuration, characterized in that: said ultrasonic probe is a convex ultrasonic probe having transducers arranged in a circular arc.

According to the ultrasonic diagnostic apparatus of the sixteenth aspect, the method of controlling an ultrasonic probe of the eighth aspect can be suitably implemented.

According to the method of controlling an ultrasonic probe and ultrasonic diagnostic apparatus of the present invention, a desired ultrasonic beam can be formed even when an acoustic line is directed obliquely with respect to an axis of symmetry of a transducer in-aperture array. Thus, image quality is improved.

The present invention may be applied to improve image quality in conducting a combination scan of linear and sector scan schemes or convex and sector scan schemes.

Further objects and advantages of the present invention will be apparent from the following description of the preferred embodiments of the invention as illustrated in the accompanying drawings.

### BRIEF DESCRIPTION OF THE DRAWINGS

FIG. 1 is an overall configuration diagram of an ultrasonic diagnostic apparatus in accordance with Example 1.

FIG. 2 is an explanatory diagram showing a portion of a transducer array and a circuit in a transmitting/receiving section corresponding to two transducers in accordance with Example 1.

FIG. 3 is a plot showing a sidelobe suppressing weight for the transducers.

FIG. 4 is a plot showing a decay value for the transducers.

FIG. 5 is a plot showing the weight for the transducers as viewed from a focus without applying the present invention.

FIG. 6 is a plot showing the weight assigned to the transducers in accordance with Example 1.

FIG. 7 is a plot showing the weight for the transducers as viewed from the focus when applying the present invention.

FIG. 8 is an explanatory diagram showing a portion of a transducer array and a circuit in a transmitting/receiving section corresponding to two transducers in accordance with Example 2.

### DETAILED DESCRIPTION OF THE INVENTION

The present invention will now be described in more detail with reference to embodiments shown in the accompanying drawings. It should be noted that the present invention is not limited to the embodiments.

### EXAMPLE 1

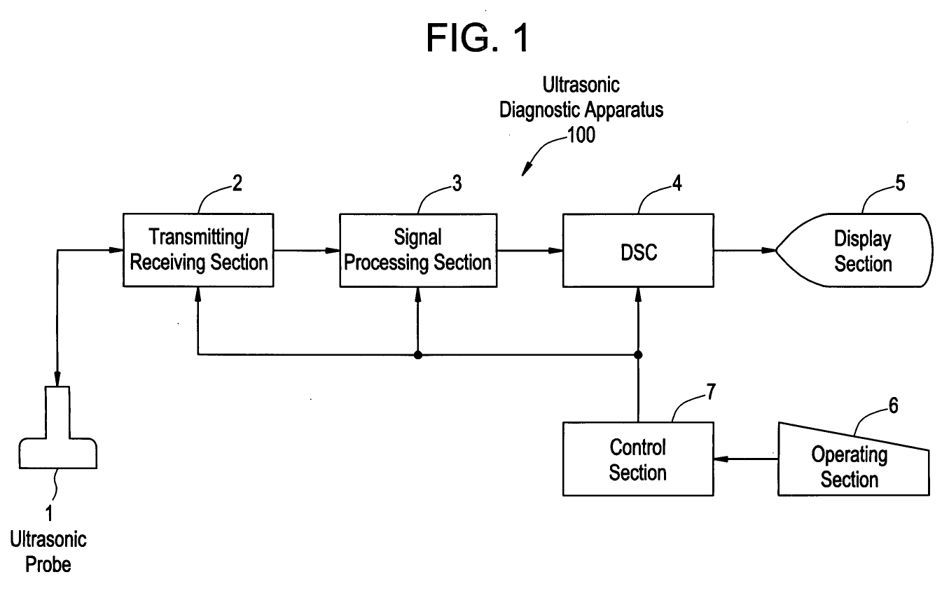

FIG. 1 is an overall configuration diagram of an ultrasonic diagnostic apparatus in accordance with Example 1.

The ultrasonic diagnostic apparatus 100 comprises an ultrasonic probe 1 having a large number of transducers arranged therein, a transmitting/receiving section 2 for driving the ultrasonic probe 1 to transmit ultrasound into a subject, receive echoes from the interior of the subject, and output received signals, a signal processing section 3 for processing the received signals to generate ultrasonic image data, a DSC (digital scan converter) 4 for controlling display of an ultrasonic image, a display section 5 for displaying an ultrasonic image, an operating section 6 for an operator supplying instructions etc., and a controlling section 7 for controlling the operation of the ultrasonic diagnostic apparatus 100.

In Example 1, a linear ultrasonic probe is assumed as the ultrasonic probe 1.

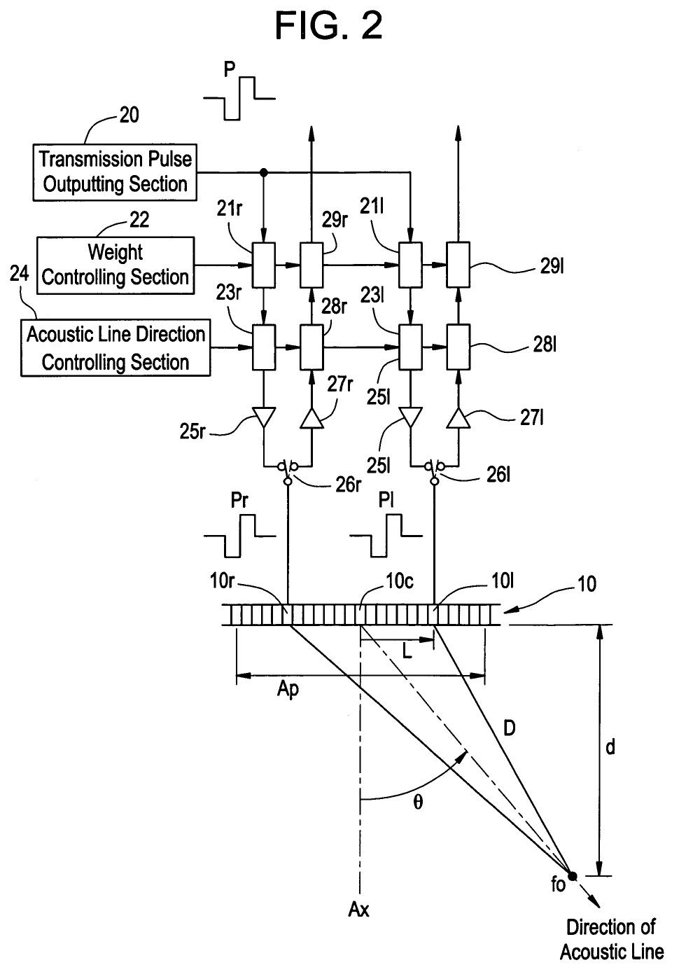

FIG. 2 is an explanatory diagram showing a portion of a transducer array 10 in the ultrasonic probe 1 and a circuit in the transmitting/receiving section 2 corresponding to two transducers 10l and 10r.

The two transducers 10l and 10r lie at positions symmetric with respect to an axis of symmetry Ax of a transducer in-aperture Ap array. The transducer 10c lies at the position of the axis of symmetry Ax.

A transmission pulse output section 20 outputs a transmission pulse P.

Amplitude/pulse-width modifying circuits 21l and 21r modify the amplitude and pulse width of the input transmission pulse P under control of a weight controlling section 22.

Transmission delay circuits 23l and 23r delay the transmission pulse P having the modified amplitude and pulse width under control of an acoustic line direction controlling section 24.

Drive circuits 251 and 25r output transducer driving pulses Pl and Pr based on the delayed transmission pulse P having the modified amplitude and pulse width.

T/R (transmission/reception) switches 261 and 26r communicate the transducer driving pulses Pl and Pr to the transducers 10l and 10r in transmission, and echo signals detected at the transducers 10l and 10r to preamplifiers 27l and 27r in reception.

The preamplifiers 27l and 27r amplify the echo signals.

Reception delay circuits 28l and 28r delay the amplified echo signals under control of the acoustic line direction controlling section 24.

Variable-gain amplification circuits 29l and 29r amplify the amplified and delayed echo signals under control of the weight controlling section 22.

The echo signals at the variable-gain amplification circuits 29l and 29r are added at an adder circuit (not shown) to form a received signal.

By controlling the delay time by the acoustic line direction controlling section 24, the position of a focusfo of an ultrasonic beam is determined. The direction of the focusfo as viewed from an intersection of the transducer array 10 and axis of symmetry Ax is the direction of an acoustic line. The angle of the direction of an acoustic line with respect to the axis of symmetry Ax in a counterclockwise direction is the angle of transmission/reception θ. The depth d of the focus fo is the distance from the intersection of the transducer array 10 and axis of symmetry Ax to the focus fo along the axis of symmetry Ax.

Representing the frequency of ultrasound as F [MHz], decay coefficient as α [dB/MHz·cm], and distance from a transducer in the aperture Ap to the focus fo as D [cm], the rate of decay At [dB] corresponding to that transducer of interest is given by the following equation:
At=F×α×D,
wherein the decay coefficient α is 0.3-0.6.

Representing the distance from the intersection of the transducer array 10 and axis of symmetry Ax to the transducer of interest as L (the distance is defined to be positive in a direction from the intersection to the focus f0), we have:
D=\sqrt{{d+(d×tan θ−L)²}},
and hence,
   At=F×α×\sqrt{{d²+(d×tan θ−L)²}}.

FIG. 4 is a plot showing the relative value of the rate of decay At of transducers, wherein F=10, α=0.5, θ=30°, d=5 [cm], and an aperture width W=[mm].

The rate of decay At of the transducer 10c is defined as 1.0.

As can be seen from FIG. 4, the rate of decay At of the transducers is asymmetric with respect to the axis of symmetry Ax (corresponding to the transducer 10c) of the transducer array.

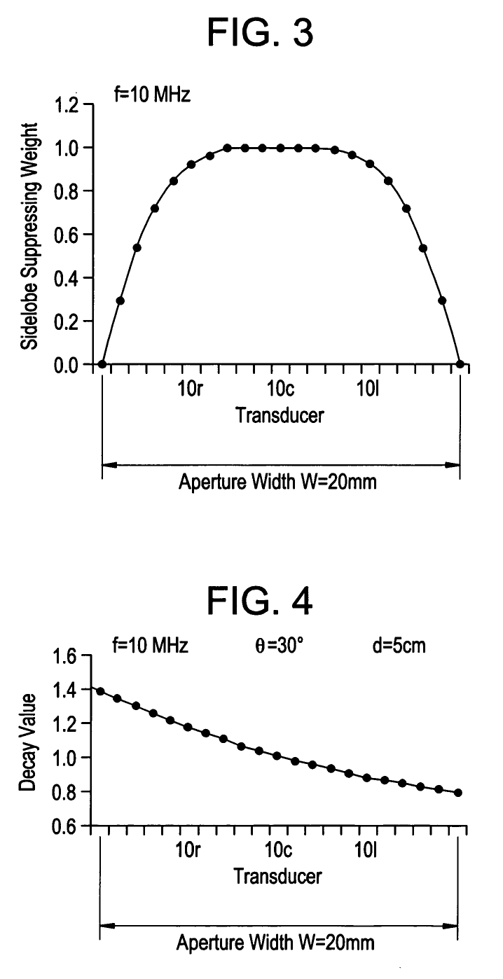

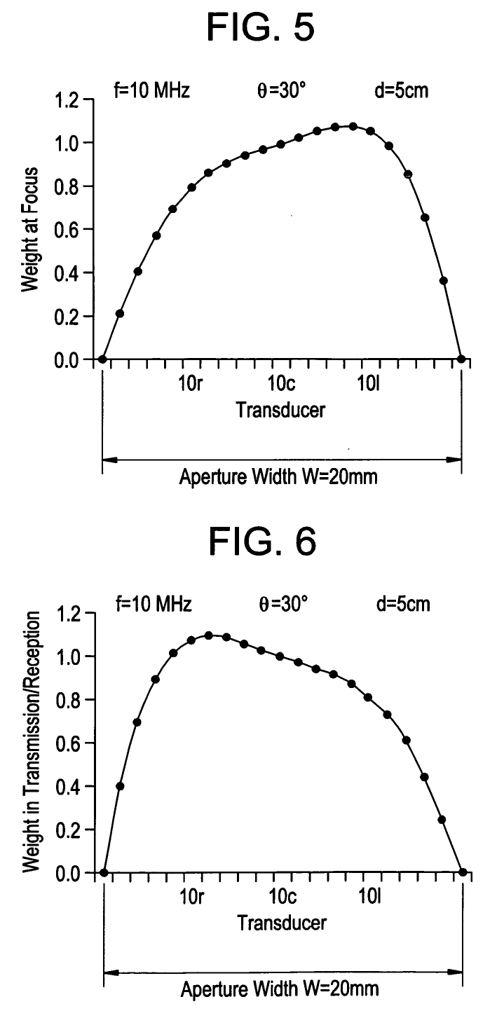

Thus, if a sidelobe suppressing weight that is symmetric with respect to the axis of symmetry of the transducer array as shown in FIG. 3 is assigned to the transducers, the weight for the transducers as viewed from the focus fo becomes asymmetric as shown in FIG. 5, and a desired ultrasonic beam cannot be formed.

The weight controlling section 22 then controls at least one of the amplitude of the transducer driving pulse, pulse width of the transducer driving pulse, and gain of the variable-gain amplification circuit, to assign the weight as exemplarily shown in FIG. 6 to the transducers so that asymmetry of the rate of decay At of the transducers is compensated.

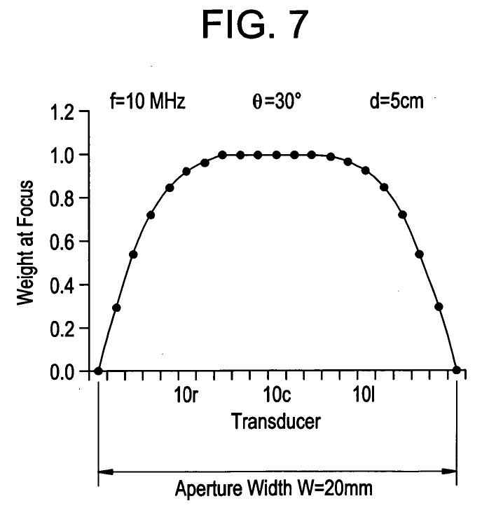

As a result, the weight for the transducers as viewed from the focus fo becomes symmetric as shown in FIG. 7, thus forming a desired ultrasonic beam.

A difference ΔAt of the rate of decay At for a transducer lying at a position symmetric with respect to the axis of symmetry Ax is given using the aforementioned equation of the rate of decay At as follows:
ΔAt=F×α×\sqrt{{d²+(d×tan θ−L)²}}−\sqrt{{d²(d×tan θ−L)²}}.

As can be seen from the equation, the asymmetry of the decay value At is larger for a higher frequency F. So the degree of asymmetry of the weight assigned to the transducers is increased for a higher frequency F.

Moreover, the asymmetry of the decay value At is larger for a larger angle of transmission/reception θ. Therefore, the degree of asymmetry of the weight assigned to the transducers is increased for a larger angle of transmission/reception θ.

According to the ultrasonic diagnostic apparatus 100 of Example 1, a desired ultrasonic beam can be formed even when an acoustic line is directed obliquely with respect to the axis of symmetry Ax of a transducer in-aperture Ap array.

### EXAMPLE 2

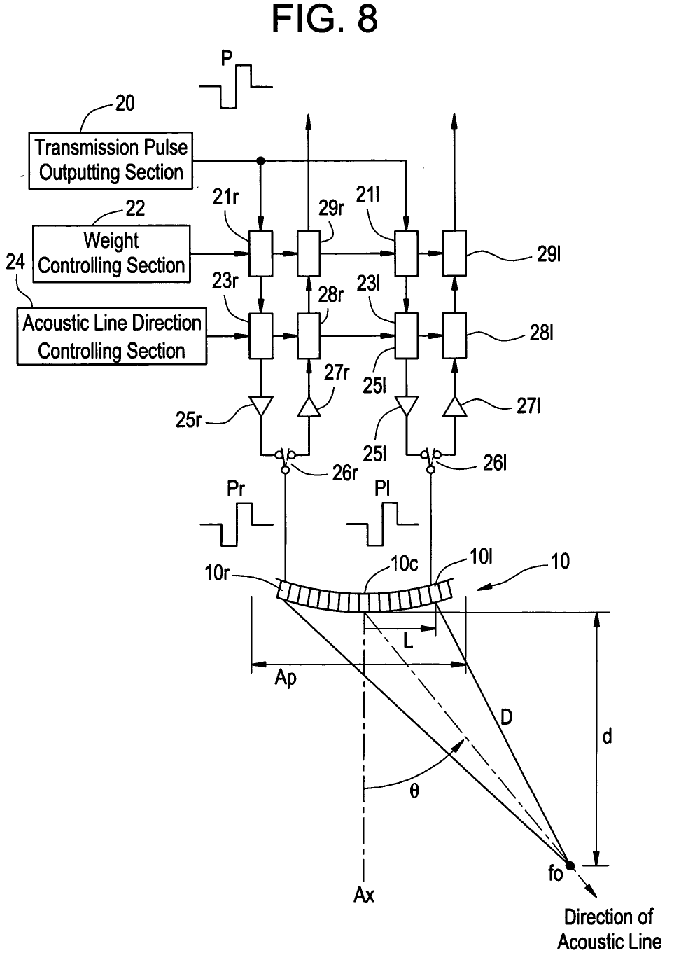

As shown in FIG. 8, the present invention can be applied, as in Example 1, to a case in which a convex ultrasonic probe is employed as the ultrasonic probe 1.

Many widely different embodiments of the invention may be configured without departing from the spirit and the scope of the present invention. It should be understood that the present invention is not limited to the specific embodiments described in the specification, except as defined in the appended claims.

1. A method of controlling an ultrasonic probe comprising the steps of: setting a transmission delay time and a reception delay time to be asymmetric for transducers lying at positions symmetric with respect to an axis of symmetry of a transducer in-aperture array to direct an acoustic line obliquely with respect to said axis of symmetry; and setting at least one of a transmission power and a reception gain corresponding to said transducers to be asymmetric.

2. The method of controlling an ultrasonic probe of claim 1, wherein at least one of a transmission power and a reception gain is decreased for a transducer having a longer transmission delay time and reception delay time.

3. The method of controlling an ultrasonic probe of claim 1, wherein the degree of asymmetry is increased for a higher frequency of ultrasound.

4. The method of controlling an ultrasonic probe of claim 1, wherein the degree of asymmetry is increased for a larger angle of the oblique direction of an acoustic line with respect to said axis of symmetry.

5. The method of controlling an ultrasonic probe of claim 1, wherein said transmission power is controlled by controlling the amplitude of a transducer driving pulse.

6. The method of controlling an ultrasonic probe of claim 1, wherein said transmission power is controlled by controlling the pulse width of the transducer driving pulse.

7. The method of controlling an ultrasonic probe of claim 1, wherein said ultrasonic probe is a linear ultrasonic probe having transducers arranged in a straight line, and said linear ultrasonic probe is used to conduct a virtual convex scan.

8. The method of controlling an ultrasonic probe of claim 1, wherein said ultrasonic probe is a convex ultrasonic probe having transducers arranged in a circular arc, and said convex ultrasonic probe is used to conduct an offset convex scan.

9. An ultrasonic diagnostic apparatus comprising: an ultrasonic probe having a plurality of transducers arranged therein; an acoustic line direction controlling device for setting a transmission delay time and a reception delay time to be asymmetric for transducers lying at positions symmetric with respect to an axis of symmetry of a transducer in-aperture array to direct an acoustic line obliquely with respect to said axis of symmetry; and a weight controlling device for setting at least one of a transmission power and a reception gain corresponding to said transducers to be asymmetric.

10. The ultrasonic diagnostic apparatus of claim 9, wherein said weight controlling device decreases at least one of a transmission power and a reception gain for a transducer having a longer transmission delay time and reception delay time.

11. The ultrasonic diagnostic apparatus of claim 9, wherein said weight controlling device increases the degree of asymmetry for a higher frequency of ultrasound.

12. The ultrasonic diagnostic apparatus of claim 9, wherein said weight controlling device increases the degree of asymmetry for a larger angle of the oblique direction of an acoustic line with respect to said axis of symmetry.

13. The ultrasonic diagnostic apparatus of claim 9, wherein said transmission power is controlled by controlling the amplitude of a transducer driving pulse.

14. The ultrasonic diagnostic apparatus of claim 9, wherein said transmission power is controlled by controlling the pulse width of the transducer driving pulse.

15. The ultrasonic diagnostic apparatus of claim 9, wherein said ultrasonic probe is a linear ultrasonic probe having transducers arranged in a straight line.

16. The ultrasonic diagnostic apparatus of claim 9, wherein said ultrasonic probe is a convex ultrasonic probe having transducers arranged in a circular arc.
